(19) United States
(12) Patent Application Publication
(10) Pub. No.: US 2016/0030713 A1
(43) Pub. Date: Feb. 4, 2016

(54) Medication Delivery Assembly

(71) Applicant: James Galinat, Vernon, CT (US)

(72) Inventor: James Galinat, Vernon, CT (US)

(21) Appl. No.: 14/449,774

(22) Filed: Aug. 1, 2014

(51) Int. Cl. A61M 25/00

(52) U.S. Cl. CPC A61M 25/0082; A61M 25/007

(57) ABSTRACT

A medication delivery assembly includes a catheter that may be inserted into the human body such as into a human bladder. The catheter is structured so a medication channel is defined in the catheter. A medication port is coupled to the catheter. The medication port is in communication with the medication channel. The medication port is penetrable so the medication port may receive a medication. The medication is deliverable into the medication channel. A dispenser is coupled to the catheter. The dispenser is in communication with the medication channel. The medication is deliverable into the dispenser. The dispenser may release the medication into the human bladder.

### BACKGROUND OF THE DISCLOSURE

### Field of the Disclosure

The disclosure relates to medication delivery devices and more particularly pertains to a new medication delivery device for delivering the medication into the human body such as in a catheterized human bladder.

SUMMARY OF THE DISCLOSURE

An embodiment of the disclosure meets the needs presented above by generally comprising a catheter that may be inserted into a human bladder. The catheter is structured so a medication channel is defined in the catheter. A medication port is coupled to the catheter. The medication port is in communication with the medication channel. The medication port is penetrable so the medication port may receive a medication. The medication is deliverable into the medication channel. A dispenser is coupled to the catheter. The dispenser is in communication with the medication channel. The medication is deliverable into the dispenser. The dispenser may release the medication into the human bladder.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a medication delivery assembly according to an embodiment of the disclosure.

FIG. 2 is a right side view of an embodiment of the disclosure.

FIG. 3 is a cross sectional view taken along line 3-3 of FIG. 2 of an embodiment of the disclosure.

FIG. 4 is a cross sectional view taken along line 4-4 of FIG. 2 of an embodiment of the disclosure.

FIG. 5 is a cross sectional view taken along line 5-5 of FIG. 2 of an embodiment of the disclosure.

### DESCRIPTION OF THE PREFERRED EMBODIMENT

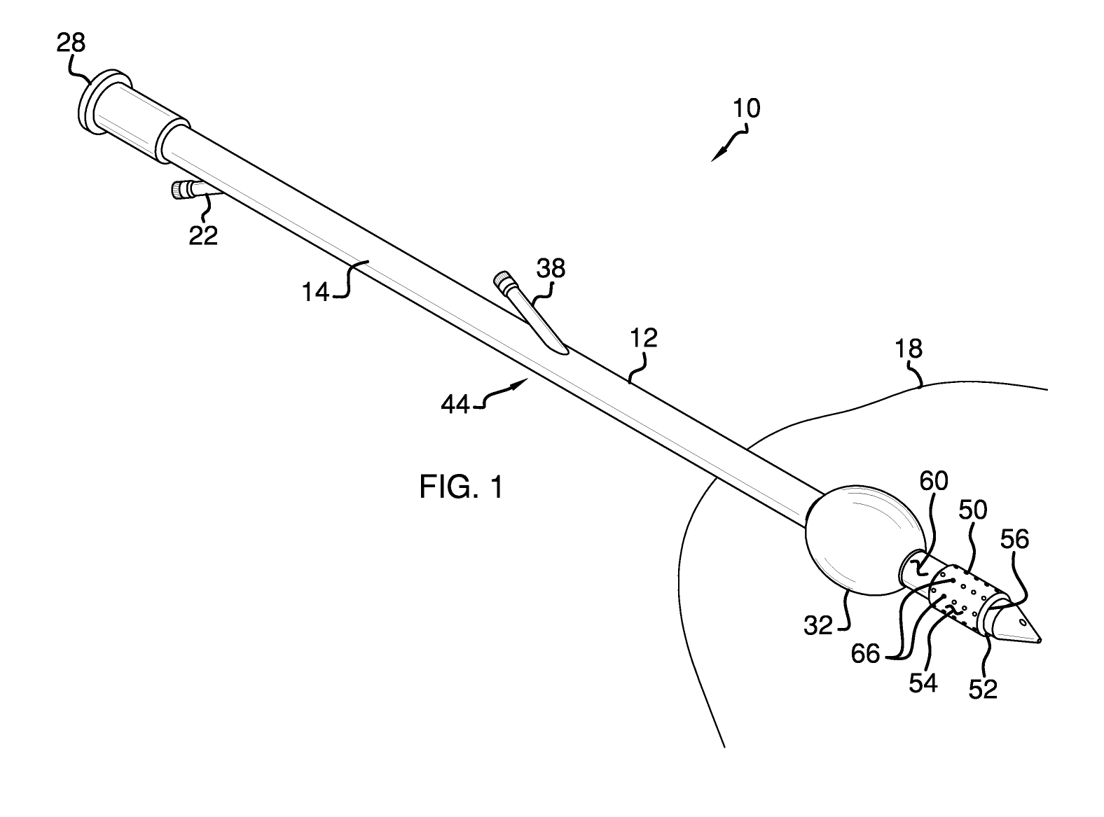

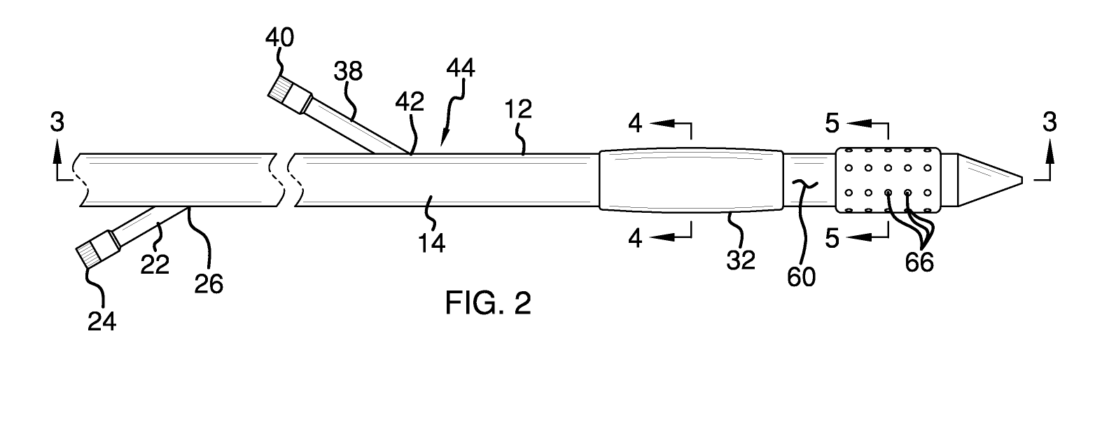

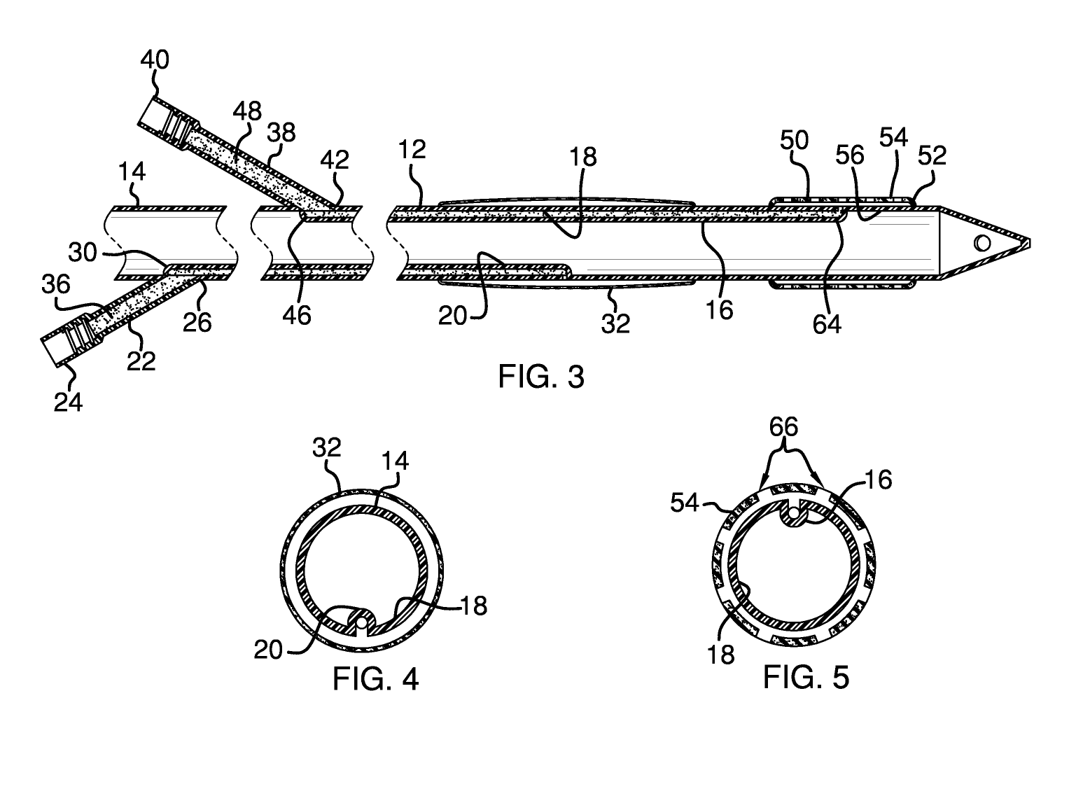

With reference now to the drawings, and in particular to FIGS. 1 through 5 thereof, a new medication delivery device embodying the principles and concepts of an embodiment of the disclosure and generally designated by the reference numeral 10 will be described.

As best illustrated in FIGS. 1 through 5, the medication delivery assembly 10 generally comprises a catheter 12. The catheter 12 has an outer wall 14. The outer wall 14 of the catheter 12 is structured so a medication channel 16 is defined on an inside surface 18 of the outer wall 14 of the catheter 12. The medication channel 16 extends longitudinally along the catheter 12. Additionally, the catheter 12 may be inserted into the human body such as into a human bladder 58. The catheter 12 may be a foley catheter or the like of any conventional design. It is to be understood also that the invention may be utilized for all situations in which an instrument is introduced into the human body.

The catheter 12 is further structured so a fluid channel 20 is defined on the inside surface 18 of the outer wall 14 of the catheter 12. The fluid channel 20 extends longitudinally along the catheter 12. A fluid port 22 is provided. The fluid port 22 has a free end 24 and a fixed end 26. Moreover, the fixed end 26 of the fluid port 22 is coupled to the outer wall 14 of the catheter 12.

The fluid port 22 is positioned proximate a draining end 28 of the catheter 12 so the fluid port 22 is fluidly coupled to a first end 30 of the fluid channel 20. A balloon 32 is coupled around the outer wall 14 of the catheter 12. The balloon 32 is positioned proximate an insertion end 34 of the catheter 12. Additionally, the balloon 32 is in fluid communication with the fluid channel 20.

The free end 24 of the fluid port 22 is penetrable so a fluid 36 may be introduced into the fluid channel 20. The fluid 36 inflates the balloon 32 after the insertion end 34 of the catheter 12 has been inserted into the human bladder 58. The balloon 32 retains the insertion end 34 of the catheter 12 in the human bladder 58.

A medication port 38 is provided. The medication port 38 has a free end 40 and a coupled end 42. The coupled end 42 of the medication port 38 is coupled to the outer wall 14 of the catheter 12. The medication port 38 is positioned proximate a middle 44 of the catheter 12. Moreover, the medication port 38 is fluidly coupled to a primary end 46 of the medication channel 16.

The free end 40 of the medication port 38 is penetrable. The medication port 38 may receive a medication 48 so the medication 48 is deliverable into the medication channel 16. The medication 48 may be a fluid antimicrobial or analgesic medication of any conventional design.

A dispenser 50 is provided. The dispenser 50 has an extraneous edge 52 extending between each of an outermost surface 54 and an innermost surface 56 of the dispenser 50. The dispenser 50 is substantially hollow between the innermost 56 and outermost 54 surfaces of the dispenser 50. Additionally, the innermost surface 56 of the dispenser 50 is coupled to an outer surface 60 of the outer wall 14 of the catheter 12.

The dispenser 50 is positioned proximate the insertion end 34 of the catheter 12. The dispenser 50 is fluidly coupled to a secondary end 64 of the medication channel 16. The medication 48 is deliverable into the dispenser 50. The outermost surface 54 of the dispenser 50 has a plurality of perforations 66 extending therethrough. Moreover, the dispenser 50 may release the medication 48 into the human bladder 58.

In use, the catheter 12 is inserted into the human bladder 58 in the convention of catheterization. The catheter 12 is retained in the human bladder 58 through the inflation of the balloon 32. The medication 48 is introduced into the human bladder 58 to combat infection or other similar side effects related to the presence of the catheter 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medication delivery assembly comprising:
a catheter configured to be inserted into a human body, said catheter being structured such that a medication channel is defined in said catheter;
a medication port coupled to said catheter such that said medication port is in communication with said medication channel, said medication port being penetrable such that said medication port is configured to receive a medication wherein the medication is deliverable into said medication channel; and
a dispenser coupled to said catheter, said dispenser being in communication with said medication channel, the medication being deliverable into said dispenser such that said dispenser is configured to release the medication into the human body.

2. The medication delivery assembly according to claim 1, further comprising said catheter having an outer wall, said outer wall of said catheter being structured such that said medication channel is defined on an inside surface of said outer wall of said catheter, said medication channel extending longitudinally along said catheter.

3. The medication delivery assembly according to claim 2, further comprising said medication port having a free end and a coupled end, said coupled end of said medication port being coupled to said outer wall of said catheter.

4. The assembly according to claim 3, further comprising said medication port being positioned proximate a middle of said catheter such that said medication port is fluidly coupled to a primary end of said medication channel.

5. The medication delivery assembly according to claim 1, further comprising said dispenser having an extraneous edge extending between each of an outermost surface and an innermost surface of said dispenser such that said dispenser is substantially hollow between said innermost and outermost surfaces of said dispenser.

6. The medication delivery assembly according to claim 5, further comprising said innermost surface of said dispenser being coupled to an outer surface of an outer wall of said catheter.

7. The medication delivery assembly according to claim 6, further comprising such that said dispenser being positioned proximate an insertion end of said catheter such that said dispenser is fluidly coupled to a secondary end of said medication channel.

8. The assembly according to claim 7, further comprising said outermost surface of said dispenser having a plurality of perforations extending therethrough.

9. A medication delivery assembly comprising:
a catheter having an outer wall, said outer wall of said catheter being structured such that a medication channel is defined on an inside surface of said outer wall of said catheter, said medication channel extending longitudinally along said catheter, said catheter being configured to be inserted into a human body;
a medication port having a free end and a coupled end, said coupled end of said medication port being coupled to said outer wall of said catheter, said medication port being positioned proximate a middle of said catheter such that said medication port is fluidly coupled to a primary end of said medication channel, said medication port being penetrable such that said medication port is configured to receive a medication wherein the medication is deliverable into said medication channel;
a dispenser having an extraneous edge extending between each of an outermost surface and an innermost surface of said dispenser such that said dispenser is substantially hollow between said innermost and outermost surfaces of said dispenser, said innermost surface of said dispenser being coupled to an outer surface of said outer wall of said catheter, said dispenser being positioned proximate an insertion end of said catheter such that said dispenser is fluidly coupled to a secondary end of said medication channel, the medication being deliverable into said dispenser; and
said outermost surface of said dispenser having a plurality of perforations extending therethrough such that said dispenser is configured to release the medication into the human body.
